(19) United States
(12) Patent Application Publication
(10) Pub. No.: US 2023/0416074 A1
(43) Pub. Date: Dec. 28, 2023

(54) TEST TANK SIMULATOR

(71) Applicant: FirstElement Fuel Inc., Irvine, CA (US)

(72) Inventors: Ghassan Sleiman, Torrance, CA (US); Andrew Youlio, SimiValley, CA (US); Tim Brown, San Juan Capistrano, CA (US); Steven Richard Mathison, Palos Verdes Estates, CA (US); Adrienne Dao, Irvine, CA (US)

(21) Appl. No.: 18/318,071

(22) Filed: May 16, 2023

Related U.S. Application Data
(60) Provisional application No. 63/342,838, filed on May 17, 2022.

(51) Int. Cl. B67D 7/08

(52) U.S. Cl. CPC B67D 7/085

(57) ABSTRACT

A tank simulator apparatus includes: a piping flowpath configured to receive a flow of a gas, the piping flowpath defining a buffer volume and including a receptacle for receiving gas flow at an upstream end thereof; a pressure transducer disposed in flow communication with the piping flowpath, the pressure transducer being configured to measure a pressure of the gas and generate a signal representative thereof; a pressure regulator disposed in flow communication with the piping flowpath and configured to maintain the pressure of the gas at a setpoint value; and an electronic controller operably connected to the pressure transducer and the pressure regulator, the electronic controller programmed to feed a time-varying setpoint value to the pressure regulator, the setpoint value following a tank profile representative of a tank having a predetermined volume.

### BACKGROUND OF THE INVENTION

This invention relates generally to gaseous fuel handling and more particularly to apparatus and methods for testing hydrogen fueling equipment.

Hydrogen filling stations for vehicles typically store hydrogen in the gas phase at high pressures, for example 45 MPa to 93 MPa. These stations include storage vessels as well as numerous pipes, valves, and fittings.

From time to time, hydrogen filling stations can require validation using end-to-end testing, that is, dispensing of hydrogen into a storage tank of a specified volume. This may be necessary, for example, when a filling station is initially commissioned, or after maintenance or repairs.

One problem with the validation process is that it requires test equipment that can be heavy, bulky, and costly, as a tank of the specified volume must be used.

### BRIEF SUMMARY

This problem is addressed by a compact tank simulator apparatus configured to produce a pressure rise characteristic simulating a tank of an arbitrary size.

### BRIEF DESCRIPTION OF THE DRAWINGS

The invention may be best understood by reference to the following description taken in conjunction with the accompanying drawing figures, in which:

FIG. 1 is a schematic diagram of a prior art hydrogen filling station;

FIG. 2 is a schematic diagram of an exemplary tank simulator apparatus; and

FIG. 3 is an example pressure vs. time graph for a tank filling process.

### DETAILED DESCRIPTION OF THE INVENTION

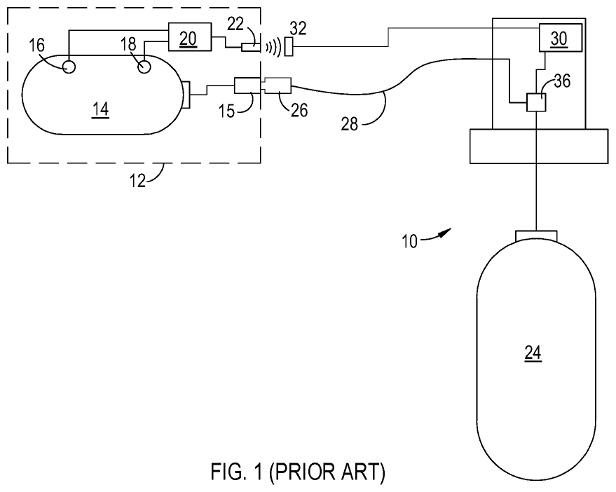

Referring to the drawings, FIG. 1 illustrates a prior art filling station 10 in conjunction with a vehicle 12. The filling station 10 is a representative example of a gaseous fuel filling apparatus. The vehicle 12 includes a gaseous fuel storage tank 14 equipped with a fill receptacle 15. The vehicle 12 includes a pressure sensor 16 operable to sense a gas pressure in the tank 14 and produce a signal representative thereof. The vehicle 12 includes a temperature sensor 18 operable to sense a gas temperature in the tank 14 and produce a signal representative thereof. The vehicle 12 includes an electronic controller 20 operable to receive the signals from the sensors and transmit that information along with other pertinent information, such as the tank volume, using a wired or wireless data transmitter 22 such as the illustrated infrared transmitter.

It is noted that the use of the vehicle is merely an illustrative example, and the tank simulator and method described herein are suitable for application to any gas storage container.

The filling station 10 includes a fuel supply 24 (e.g. hydrogen). The fuel may be stored as liquid, low-pressure gas, or high-pressure gas, and is dispensed in gaseous form through a nozzle 26 which is disposed at a distal end of a fill hose 28 and which is configured to be coupled to the fill receptacle 15. It will be understood that a filling station 10 of this type may include conventional ancillary equipment for handling the fuel such as heat exchangers, pumps, compressors, and/or valves. The filling station 10 includes an electronic controller 30.

The controller 30 includes one or more processors capable of executing ladder logic, programmed instructions, or some combination thereof. For example, it may be a general-purpose microcomputer of a known type, such as a PC-based computer, or may be a custom processor, or may incorporate one or more programmable logic controllers (PLC).

The filling station 10 is equipped with a wired or wireless data receiver 32 configured to receive data from the data transmitter 22 of the vehicle 12, such as the illustrated infrared receiver mounted on the nozzle 26. The data receiver 32 is operably connected to the controller 30.

The filling station 10 includes at least one control device operable to affect some aspect of the flow of gaseous fuel. One example of a control device is a controllable valve, shown schematically at 36. This could be, for example a flow metering valve operated by an actuator operably connected to the controller 30 such that the controller 30 can change the flow rate through the flow metering valve.

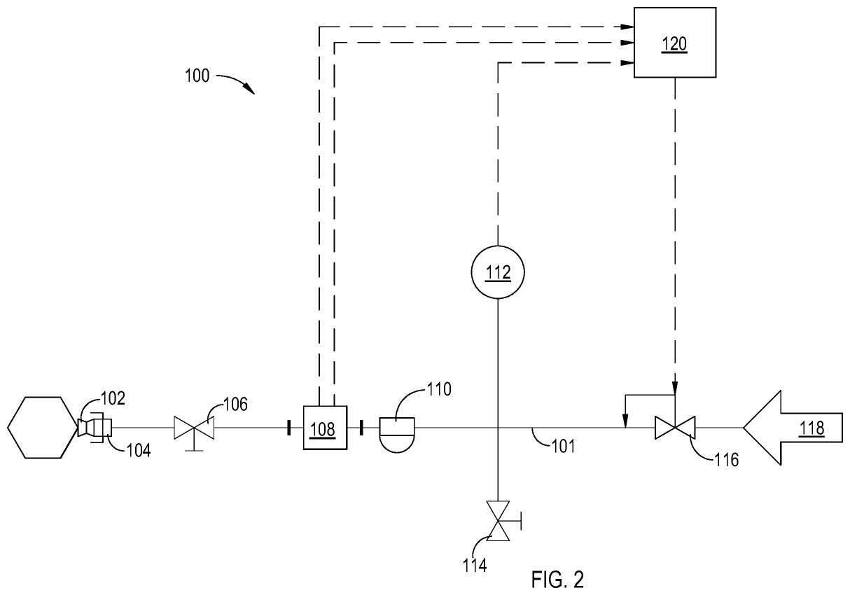

From time to time it is necessary to calibrate or validate the operation of the filling station 10. FIG. 2 illustrates an exemplary a tank simulator apparatus 100 suitable for this purpose.

It will be understood that the operating components of the apparatus 100 are interconnected along a flowpath defined by piping or tubing suitable for flowing pressurized gaseous hydrogen. This piping flowpath is labeled 101 generally. In this single-line diagram, fluid connections are represented by single lines, and data and/or control connections are represented by dashed lines.

At the upstream end of the piping flowpath 101, the simulator apparatus 100 includes a hydrogen receptacle 102 for receiving gas flow. It may be of the same general type as the fill receptacle 15 described above. The hydrogen receptacle 102 is configured to be coupled to the nozzle 26 of the filling station 10. A data transmitter 104 corresponding to the data transmitter 22 described above is associated with the hydrogen receptacle 102, for example it may be physically mounted in close proximity to it. The data transmitter 104 may operate in the infrared band.

A first valve 106 (alternatively referred to as a trim valve) is coupled downstream of the hydrogen receptacle 102. The needle valve 106 is operable to provide a variable restriction to hydrogen flow. One example of a valve suitable for this purpose is a needle valve.

A flowmeter 108 is coupled downstream of the needle valve 106. The flowmeter 108 is operable to sense a mass flow rate of hydrogen and generate a signal representative thereof. It is also operable to sense a temperature of hydrogen and generate a signal representative thereof. Such devices are commercially available. Alternatively, flowrate and temperature may be detected using separate sensors.

The apparatus 100 has a defined buffer volume. In the illustrated example, a buffer tank 110 is coupled downstream of the flowmeter 108. The buffer tank 110 has a small volume, for example about 1 liter. In this example configuration, the buffer tank 110 represents the majority of the system's buffer volume. Alternatively, the buffer volume could be defined by piping.

A pressure transducer 112 is coupled downstream of the buffer tank 110. The pressure transducer 112 is operable to sense a pressure of hydrogen and generate a signal representative thereof. Such devices are commercially available.

A second valve 114 (alternatively referred to as a vent valve) is coupled downstream of the buffer tank 110. The second needle valve 114 is operable to vent hydrogen to the atmosphere at a user-selectable rate. One example of a valve suitable for this purpose is a needle valve.

A hydrogen pressure regulator 116 is coupled downstream of the buffer tank 110. The hydrogen pressure regulator 116 is configured as a “backpressure regulator” operable to maintain gas pressure upstream at a set point pressure. In this particular example, the hydrogen pressure regulator 116 is pneumatically operated (that is, the reference pressure is compressed air) and is electronically controlled. Suitable regulators for this purpose are commercially available from Emerson Electric Co., St. Louis, MO 63136 USA. Pneumatic operation is just one example of a pressure regulator operating method. Other types of regulators are available, including regulators that do not require a separate reference source, such as electrically actuated regulators.

The hydrogen pressure regulator 116 is the furthest downstream component in the hydrogen piping flowpath 101. It is configured so that excess hydrogen is vented to atmosphere.

A pressurized air supply 118 is coupled to the hydrogen pressure regulator 116, for the purpose of supplying the reference pressure.

The apparatus 100 includes an electronic controller 120. The controller 120 includes one or more processors capable of executing ladder logic, programmed instructions, or some combination thereof. For example, it may be a general-purpose microcomputer of a known type, such as a PC-based computer, or may be a custom processor, or may incorporate one or more programmable logic controllers (PLC).

The controller 120 receives inputs of a representative hydrogen temperature and flow rate from the flowmeter 108, and hydrogen pressure from the pressure transducer 112. The controller 120 is operable to control the hydrogen pressure regulator 116. More specifically, the controller 120 is programmed to calculate a pressure setpoint that is a function of the other input conditions, i.e., the mass flow rate and representative hydrogen temperature for a predetermined tank volume. The controller 120 feeds the calculated pressure setpoint signal to the hydrogen pressure regulator 116.

The controller 120 may be programmed with multiple tank profiles, consisting of different tank volumes and different hydrogen temperature profiles.

This controller function enables the apparatus 100 to simulate a filling process for various sizes of tanks, including large volume tanks, utilizing only a small volume buffer tank. The simulation process is carried out by controlling the pressure rise over time in the buffer tank to mimic the behavior of a larger tank.

It will be understood that, following the ideal gas law (PV=nRT) with a compressibility factor, or a similar pressure mass relationship, for a fixed volume container the gas pressure increases with increasing mass contained in the tank. Furthermore, for a given flow rate into the tank, the pressure in the tank will increase faster for a smaller volume of tank.

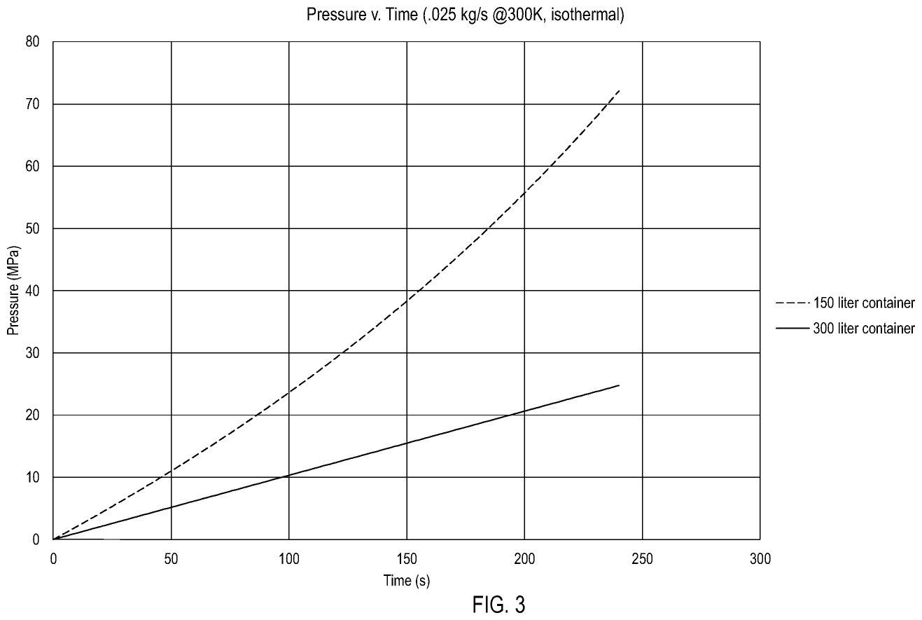

This characteristic is demonstrated in FIG. 3 which illustrates a computed pressure vs. volume at a fixed flow rate, starting from an empty tank (further accounting for gas compressibility). For this example, the process was modeled as isothermal at a gaseous hydrogen flow rate of 0.025 kg/s. The upper line shows, for example, that at an elapsed time of 100 seconds, a 150 liter tank reaches a pressure of approximately 24 MPa, while at an elapsed time of 100 seconds, a 300 liter tank reaches a pressure of approximately 11 MPa.

The apparatus 100 is constructed such that, when the pressure exceeds the setpoint of the hydrogen pressure regulator 116, hydrogen is vented to atmosphere. The maximum venting rate of the apparatus 100 is sufficient to enable the pressure in the buffer tank 110 to be maintained or lowered even as hydrogen flows in from the filling station 10.

The apparatus 100 would be operated by coupling the hydrogen receptacle 102 to the nozzle 26. An appropriate tank profile would then be selected in the controller 120. The tank fill simulation begins by flowing hydrogen into the buffer tank 110. As the buffer tank 110 fills, the controller 120 controls the pressure rise therein by supplying the hydrogen pressure regulator 116 with the appropriate setpoint at each moment in time, following the selected tank profile. As far as the fuel station 10 is concerned, the behavior of the downstream equipment will be exactly as if a tank of the simulated size were being filled.

The apparatus 100 may be provided in a single compact enclosure. For example, the entire apparatus may be approximately the size of a small piece of luggage. This makes it easily portable. Alternatively, some or all of the components could be incorporated into the filling station 10 itself.

This apparatus has certain advantages. It permits testing and validation of dispensing equipment using relatively lightweight and compact testing equipment that can be inexpensive and easily transported. It can also permit testing using a substantially lower amount of hydrogen than would otherwise be required.

The foregoing has described a tank simulator apparatus and method. All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings) may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of the foregoing embodiment(s). The invention extends to any novel one, or any novel combination, of the features disclosed in this specification (including any accompanying claims, abstract and drawings), or to any novel one, or any novel combination, of the steps of any method or process so disclosed.

What is claimed is:

1. A tank simulator apparatus, comprising:
a piping flowpath configured to receive a flow of a gas, the piping flowpath defining a buffer volume and including a receptacle for receiving gas flow at an upstream end thereof;
a pressure transducer disposed in flow communication with the piping flowpath, the pressure transducer being configured to measure a pressure of the gas and generate a signal representative thereof;
a pressure regulator disposed in flow communication with the piping flowpath and configured to maintain the pressure of the gas at a setpoint value; and
an electronic controller operably connected to the pressure transducer and the pressure regulator, the electronic controller programmed to feed a time-varying setpoint value to the pressure regulator, the setpoint value following a tank profile representative of a tank having a predetermined volume.

2. The tank simulator apparatus of claim 1, further comprising a flowmeter disposed in flow communication with the piping flowpath, the flowmeter being operable to sense a mass flow rate of the gas and generate a signal representative thereof.

3. The tank simulator apparatus of claim 2, further comprising a temperature sensor disposed in flow communication with the piping flowpath, the temperature sensor being operable to sense a temperature of the gas and generate a signal representative thereof.

4. The tank simulator apparatus of claim 1, wherein the buffer volume includes a buffer tank disposed in flow communication with the piping flowpath.

5. The tank simulator apparatus of claim 1, wherein:
the pressure regulator is configured as a backpressure regulator;
the pressure regulator is the furthest downstream component in the piping flowpath; and
the pressure regulator is configured to vent excess gas to atmosphere.

6. The tank simulator apparatus of claim 1, wherein:
the pressure regulator is pneumatically operated; and
a pressurized air supply is coupled to the pressure regulator.

7. The tank simulator apparatus of claim 1, further comprising a trim valve disposed in flow communication with the piping flowpath, downstream of the receptacle and upstream of the pressure transducer, the trim valve being operable to provide an adjustable restriction to gas flow.

8. The tank simulator apparatus of claim 1, further comprising a vent valve disposed in flow communication with the piping flowpath, upstream of the pressure regulator, the vent valve being operable to vent gas to the atmosphere.

9. A method of testing a gaseous fuel filling apparatus, comprising:
coupling a tank simulator apparatus to the filling apparatus, the tank simulator apparatus defining a buffer volume;
flowing a gas from the filling apparatus to the tank simulator apparatus; and
while flowing the gas, selectively venting the gas from the buffer volume to atmosphere at a flowrate varying with time, such that a pressure rise of the gas over time follows a tank profile representative of a tank having a predetermined volume which is greater than the buffer volume.

10. The method of claim 9, wherein the tank simulator apparatus comprises:
a piping flowpath defining the buffer volume and including a receptacle for receiving gas flow at an upstream end thereof;
a pressure transducer disposed in flow communication with the piping flowpath, the pressure transducer being configured to measure a pressure of the gas and generate a signal representative thereof;
a pressure regulator disposed in flow communication with the piping flowpath and configured to maintain the pressure of the gas at a setpoint value; and
an electronic controller operably connected to the pressure transducer and the pressure regulator, wherein the flowrate is varied by using the electronic controller to feed a time-varying setpoint value to the pressure regulator.

11. The method of claim 10, wherein the tank simulator apparatus comprises a flowmeter disposed in flow communication with the piping flowpath, the method further comprising using the flowmeter to sense a mass flow rate of the gas and send a signal representative thereof to the electronic controller.

12. The method of claim 11, wherein the tank simulator apparatus comprises a temperature sensor disposed in flow communication with the piping flowpath, the method further comprising using the temperature sensor to sense a temperature of the gas and send a signal representative thereof to the electronic controller.

13. The method of claim 10, wherein the buffer volume includes a buffer tank disposed in flow communication with the piping flowpath.

14. The method of claim 10, wherein:
the pressure regulator is configured as a backpressure regulator;
the pressure regulator is the furthest downstream component in the piping flowpath; and
the pressure regulator is configured to vent excess gas to atmosphere.

15. The method of claim 10, wherein:
the pressure regulator is pneumatically operated; and
a pressurized air supply is coupled to the pressure regulator.

16. The method of claim 10, wherein the tank simulator apparatus comprises a trim valve disposed in flow communication with the piping flowpath, the method further comprising using the trim valve to provide a variable restriction to hydrogen flow.
